(19) United States
(12) Patent Application Publication
(10) Pub. No.: US 2018/0230174 A1
(43) Pub. Date: Aug. 16, 2018

(54) Microwave Assisted Hydrothermal Pre-Treatment of Cellulosic Material to Produce Fermentable Mixtures

(71) Applicant: University of York, Heslington, York (GB)

(72) Inventors: Vitaliy Budarin, Heslington (GB); James Hanley Clark, Heslington (GB); Jiajun Fan, Heslington (GB); Mark Gronnow, Heslington (GB)

(73) Assignee: University of York, Heslington, York (GB)

(21) Appl. No.: 15/545,046

(22) PCT Filed: Jan. 20, 2016
(86) PCT No.: PCT/GB2016/050116

§ 371 Date: Jul. 20, 2017

(30) Foreign Application Priority Data
Jan. 20, 2015 (GB) 1500894.9

(51) Int. Cl. C07H 1/08; C07H 3/02; C07H 3/06; C13K 1/02; C12P 7/10; H05B 6/80

(52) U.S. Cl. CPC C07H 1/08; C07H 3/02; C07H 3/06; C12P 2201/00; C12P 7/10; H05B 6/80; C13K 1/02

(57) ABSTRACT

There is described a method of transformation of a cellulosic material, such as, lignocellulosic biomass material and/or cellulose, into a directly fermentable saccharide containing mixture wherein said method of transformation comprises the microwave assisted hydrothermal treatment of the cellulosic material.

### FIELD OF THE INVENTION

The present invention relates to a novel process of bio-refinery for processing of cellulosic materials, such as, lignocellulosic biomass material and/or cellulose, to produce a fermentable organic mixture.

More particularly, the invention relates to a bio-refinery for processing of cellulosic materials, such as, lignocellulosic biomass material and/or cellulose, via microwave assisted hydrothermal low temperature treatment for the production of a fermentable organic mixture.

Generally, the bio-refinery processing comprises a microwave assisted hydrothermal process conducted at above atmospheric pressure and ambient temperatures. The bio-refinery processing does not require acid, alkaline or such like additives to assist in the breakdown of cellulosic materials to a fermentable mixture.

### BACKGROUND TO THE INVENTION

In the 21⁴ century the demand for new sources of feedstocks for refinery processing has substantially increased due to depleting easily available oil resources and the ever increasing pressure of global warming¹⁻². Of the potential feedstocks, sugars from plants present an interesting route; not for their direct use, but after biological processing to produce alcohols for use as fuels³. However, sugars from plants are also commonly a food source, hence making it controversial to use them.

Hence, cellulosic materials that are abundant and readily available source of biomass on the planet, have the potential to be converted to non-food competing sugars. As such, methodologies to optimise its breakdown to simple sugars are highly desirable⁴⁻⁵. As a result of this many technologies and approaches have been examined/explored; these include enzymatic, catalytic and thermal methods of biomass processing⁶⁻¹³. However, each approach has significant limitations. For example, enzymatic depolymerisation requires significant residence times and high dilution ratios, but gives highly selective products; whilst thermal processing is limited by poor thermal conductivities and thermal efficiencies¹⁴⁻¹⁵. Alongside this, the high temperatures required in thermal processing often lead to the production of breakdown products of sugars; anhydrosugars and secondary decomposition products; such as phenols and hydroxymethylfurfural (HMF)¹⁶.

However, hydrothermal processing of cellulosic materials offers a potentially less aggressive form of thermal processing, breaking down cellulosic materials into simple sugars using low temperature and pressure, utilising water as the medium.

Current hydrothermal technologies use sub-critical water due to the special properties. Subcritical conditions exist between waters atmospheric boiling point and 374° C.¹⁷. It exhibits the properties of low viscosity and high solubility for organic substances¹⁸. These enable fast, homogenous reactions to take place¹⁹. When in its subcritical condition, water demonstrates significant changes in its, density¹⁸, dielectric constant and hydrocarbon solubility²⁰.

Therefore, the processing of biomass by hydrothermal means offers a number of possible advantages over other methods. These include, but are not limited to:
high throughput;
high specificity;
high energy efficiency; and
high separation efficiency.

Hydrothermal processing also offers the ability to use a wide range of biomass waste materials, including mixed feedstocks for the production of direct replacements for existing fuels, with little need to maintain specialized microbial cultures or enzymes²¹. In addition, because of the high temperature involved, biofuels produced may potentially be free of biologically active microorganisms or compounds, including, for example, bacteria and viruses²¹.

However, the greatest disadvantage of high temperature/pressure water treatment is corrosion, which is a problem for all sub-critical water systems. Special materials for vessel linings and tubing are needed to resist the highly reactive chemical species generated during the process. These challenges demand superior engineering and design expertise for all system components. The water and process streams must both be pumped to high initial pressures under exact flow and pressure control. The heat exchangers are subjected to high heat transfer rates at high temperatures, but must maintain precise temperature control. The reaction vessel requires accurate temperature, pressure and flow control. The vessel must seal reliably and be leak free each time it is used.

During conventional hydrothermal treatment of lignocellulosic biomass it is often necessary to perform acid pre-treatment of the lignocellulosic biomass. This can be performed by two approaches, either using concentrated acids at lower temperatures or diluted acids at higher temperatures. This pre-treatment process is used to solubilise hemicellulose, break down the lignin and decrease cellulose crystallinity so that the biomass is more susceptible to subsequent enzymatic hydrolysis.

Concentrated acid such as sulphuric acid, hydrochloric acid or trifluoroacetic acid (TFA) can be used for biomass pretreatment²². During the treatment, the milled raw materials can be treated with concentrated acid at a temperature of less than 100° C. The concentrated acid can efficiently remove lignin and hemicellulose whilst also hydrolysing some cellulose to glucose. Nevertheless, the process requires large amounts of concentrated acid which is highly corrosive. Therefore, acid recovery and the use of specialist reactors are necessary to make the method economically feasible²³. Comparatively, dilute acid (<4 wt. %) hydrolysis is regarded as an efficient and inexpensive pre-treatment method²⁴⁻²⁵. However, all acid treatments require neutralisation after processing, thus creating large amounts of salts.

However, one drawback of acid pre-treatment is the formation of by-products or residues that inhibit the downstream fermentation process. These inhibitors include organic acids, furan derivatives and phenolic compounds. If concentrations of these are too great, detoxification procedures for the removal of these are required prior to downstream fermentation²⁶⁻²⁷.

The further process of alkaline hydrolysis is the most commonly used chemical pre-treatment method. It primarily removes lignin and some hemicellulose from lignocellulosic biomass. Pre-treatment of biomass with alkalis causes saponification of intermolecular ester bonds that crosslink lignin and xylem, this subsequently leads to delignification²⁸⁻²⁹. Sodium hydroxide is the most commonly used alkali due to its low cost. This form of pre-treatment is more effective towards lignocellulosic biomass with lower lignin content such as hardwood, herbaceous crops, and agricultural residues in comparison to those with higher lignin content such as softwood³⁰.

The process of alkaline hydrolysis is comparatively mild compared to acid pre-treatment and can be carried out in a batch mode. This pre-treatment process involves, for example, spraying alkali onto biomass and soaking it from hours to days at ambient temperature. The reaction time can greatly be reduced at elevated temperature. Xu and co-workers reported³¹ the use of dilute sodium hydroxide (1%) for the pre-treatment of switchgrass to efficiently reduce the lignin content by 85.8% at 121° C. in 1 hour, 77.8% at 50° C., and 62.9% at 21° C. for 48 hours. At the end of the process, the pre-treated biomass was recovered by filtration and neutralised before further processing.

Unlike acid hydrolysis, alkaline hydrolysis is a milder process and thus sugar degradation to furfural, HMF and organic acids is reduced²². Moreover, caustic salts such as calcium carbonate can be recovered from the aqueous liquid/solution generated from the system as insoluble calcium carbonate by neutralizing it with inexpensive carbon dioxide²². In addition, since the process can be conducted at ambient temperature and pressure the energy requirement of the process is low. However, a disadvantage of this method is the use of corrosive chemicals and its associated operating and environmental issues.

At present, conventional hydrothermal treatment is less energy efficient and does not cause sufficient breakdown of cellulosic structure to produce sufficient fermentable products to make it economically viable.

Fan, J. et al, “Direct Microwave-Assisted Hydrothermal Depolymerization of Cellulose”, J. Am. Chem. Soc. 2013, 135, 11728-11731, describes an investigation of the interaction of microwave irradiation of microcrystalline cellulose and attempts to explain the dependence of the selectivity/yield of glucose on the applied microwave density; the observed high glucose to HMF ratio; and the influence of the degree of cellulose crystallinity on the results of the hydrolysis process.

International Patent Application No. WO 2014/122439 (University of Bath) describes yeasts for biofuel production. In particular, WO 2014/122439 describes a method of obtaining oil from yeast pulcherimma cells, e.g. Metschnikowia pulcherimma and the use of pulcherimma cells for production of oleaginous biomass. The method described therein comprises providing the yeast with at least one nitrogen and/or sulphur source, and at least one carbon source, the carbon source being selected from glycerol, lignocellulose, sugar, polysaccharides, oligosaccharide, waste water, waste foods, agricultural waste or energy crops.

Therefore, there is a need for a method of processing of cellulosic material, such as lignocellulosic biomass material and/or cellulose, which is capable of producing sugars which are in a form that can be economically fermented. The hydrothermal processing of lignocellulosic biomass would be preferable, as it could eliminate the need for pre-treatments and post-treatments, required prior to downstream fermentation.

In particular, there is a need for a method of processing of cellulosic material, such as lignocellulosic biomass material and/or cellulose, to provide a carbon source low in fermentation inhibitors.

### SUMMARY TO THE INVENTION

One of the possibilities to overcome the aforementioned problems, which offers an advantageous alternative to conventional thermal processing, is the combination of hydrothermal conditions with microwave irradiation. Therefore, microwave heating presents a potentially faster, more efficient and selective hydrothermal method for the pre-treatment of biomass before biological processing, e.g. fermentation. Furthermore, microwave assisted hydrolysis takes place at much lower temperatures (around 200° C.), significantly reducing the pressure of the process from 9.8 MPa down to no more than 3 MPa. This could largely reduce equipment costs.

Herein, there is described a novel hydrothermal process for the conversion of cellulosic materials, such as, lignocellulosic biomass material and/or cellulose, into fermentable mixtures using low temperature hydrothermal microwave treatment.

Furthermore, the present invention proves that the hydrothermal microwave approach does not require any additives (e.g. acid/base), which reduces downstream treatment and corrosion to the infrastructure.

The present invention describes a single step process for cellulosic materials, such as, lignocellulosic biomass material, including lignocellulosic waste materials and/or cellulose whereby fermentable mixtures containing such chemicals as saccharides, e.g. glucose, cellobiose, fructose, arabinose etc. are produced from the direct hydrolysis of cellulosic materials under hydrothermal conditions; above ambient temperature and pressure assisted by the use of microwave irradiation.

Thus, according to a first aspect of the present invention there is provided a method of transformation of a cellulosic material into a directly fermentable saccharide containing mixture wherein said method of transformation comprises the microwave assisted hydrothermal treatment of the cellulosic material.

In all aspects of the invention herein described the cellulosic material may comprise a lignocellulosic biomass material and/or cellulose. The cellulose may optionally be pure cellulose. By the term “pure cellulose” is meant cellulose that has generally been separated from other plant natural products, such as hemicellulose and/or lignin and typically has a purity of 90% w/w or more, e.g. 95% w/w or more.

It is a particular aspect of the present invention that the directly fermentable saccharide containing mixture, prepared by the transformation of a cellulosic material, such as, lignocellulosic biomass material and/or cellulose, comprises no or very low levels of toxins and/or inhibitors, typically from about 0 g/L (none detectable) to about 15 g/L, preferably from about ≤0.1 g/L to about 5 g/L, more preferably from about ≤0.1 g/L to about 3 g/L). By the term “very low levels of toxins and/or inhibitors” is meant a level of toxins and/or inhibitors that are sufficiently low so as to permit direct fermentation, or other biological treatment, to take place without the need for an additional processing step to remove such toxins and/or inhibitors.

The saccharides will generally comprise monosaccharides, disaccharides and oligosaccharides, lignin, cellulose and/or hemi-cellulose; and mixtures thereof. Exemplary monosaccharides, include, but shall not be limited to, such as glucose, galactose, mannose, fructose, sorbose, allose, talose, gulose, altrose, idose, xylose, arabinose, ribose, and lyxose, or oligosaccharides such as sucrose, trehalose, lactose, maltose, cellobiose, raffinose, and cellotriose.

In addition, according to the present invention there is provided a directly fermentable saccharide containing mixture, prepared by the transformation of a cellulosic material, comprises sufficient nutrients so as to permit direct fermentation, or other biological treatment, to take place.

The distinct lack of any further additives, such as acid or alkaline catalysts during the process result in the production of a fermentable aqueous mixture which is free from the inhibiting effects of such additives; but also contains sufficient natural components, such as micronutrients which were originally contained within the lignocellulosic material. Through careful control of the process variables it is also possible to minimise the amount of inhibiting compounds. This negates the need for further processing of the final aqueous mixture to remove such inhibiting chemicals; allowing for the direct biological processing of the aqueous mixture.

The present invention provides a novel process for the conversion of cellulosic material and water to a fermentable mixture through microwave hydrothermal treatment without additives. Thus, a method of directly producing an aqueous fermentable mixture is provided, wherein the said method comprises of the microwave assisted hydrothermal conversion of cellulosic material takes place.

The method of hydrothermal processing of the present invention is widely applicable especially suited to use in conjunction with wet waste streams, thus avoiding the necessity to dry cellulosic materials prior to their hydrothermal processing.

An important aspect of this invention is that the transformation of the cellulosic material, i.e. the production of a fermentable mixture, can be achieved in an environment that is substantially free of acid/alkali/ionic liquid. The liquid mixture contains monosaccharides, oligosaccharides and a low amount of inhibitors/toxins, the ratio between all the compounds in the mixture are able to be used in fermentation, or other biological processes to produce bio-products, such as biofuels.

The lignocellulosic biomass material desirably comprises organic matter that is available on a renewable basis. Lignocellulosic biomass material comprises one or more of forest and/or mill residues, agricultural crops and wastes, food wastes, wood and wood wastes, agricultural waste, animal wastes, livestock operation residues, aquatic plants, fast-growing trees and plants, and municipal and industrial wastes. Preferably, the lignocellulosic material comprises waste in its origin, such as agricultural waste, forestry residue or waste paper; and mixtures thereof.

In the method of the invention, the lignocellulosic material may be combined with water prior to the material being subjected to microwave energy. Desirably, the solids-to-water ratio may vary and may be varied from 1:0.1 w/w or 1:50 w/w; this is dependent, inter alia, the type of lignocellulosic biomass used, the target fermentable mixture wanted to be achieved, etc. Alternatively, pre-existing water present in the lignocellulosic material may be utilised. Thus, the method may comprise exposing a mixture of lignocellulosic material and water to microwave energy. Alternatively, the method may comprise the microwave steam distillation of lignocellulosic material in the absence of additional water. In a further alternative, the method may comprise the microwave assisted hydrothermal treatment of lignocellulosic material in the absence of additional water. Any of the aforementioned methods may comprise multiple steps or may comprise a single step.

Microwave irradiation is defined as “electromagnetic irradiation in the frequency range of about 0.3 to about 300 GHz. Specialised chemistry microwave reactors operate at about 0.915 GHz to about 2.45 GHz. The microwave irradiation power may vary from about 100 W to about 10 MW. The method of the invention may comprise to repeat processing of the cellulosic material by microwave assisted hydrothermal treatment.

The method of the invention may be carried out at a variety of temperatures heating from ambient or elevated temperature to greater temperatures. The temperature and pressure of the hydrolysis process may vary depending, inter alia, upon the type/types of cellulosic material used, the pressure that is sought to be attained, etc. In a particular aspect, the method of the invention may be carried out at elevated temperature and/or pressure. The method may be carried out at a temperature of from about 20° C. to about 300° C. In particular, the temperature may vary from ambient to less than or equal to 100° C. initially, and the temperature may then be raised to from about 100° C. to about 250° C., with corresponding changes in pressure taking place within the reactor vessel.

Once maximum hydrolysis temperature is achieved a holding time may be applied to the hydrolysis mixture from about 0.1 seconds to about 24 hours.

It will be understood by the person skilled in the art that the fermentable saccharide containing mixture may be utilised directly in a fermentation step. However, it will also be understood by the person skilled the art that the isolation of such monosaccharides and oligosaccharide from the transformation of the cellulosic material may be possible. Such compounds may be isolated simultaneously, sequentially or separately. Thus, the present invention further provides an integrated biorefinery approach to the isolation of components herein before described from cellulosic materials. The method of invention is also advantageous in that, inter alia, it allows for the isolation of one or more monosaccharides such as glucose, fructose, galactose etc. from the transformation of cellulosic material after being exposed to microwave irradiation under hydrothermal conditions as herein described. In addition, the transformation of a cellulosic material may produce one or more decomposition products which may optionally be isolated, such as, one or more of 5-hydroxymethylfurfural (HMF), levoglucosan and levoglucosenone; and mixtures thereof.

Thus, according to a further aspect of the invention there is provided a decomposition product of a monosaccharide or oligosaccharide prepared by the method as herein described. Such decomposition products may be selected from the group consisting of one or more of rhamnose, galactose, mannose, xylose, fructose, glucose, sucrose, cellobiose, hydroxymethylfurfural, levoglucosenone and furfural; and mixtures thereof. Preferably, the decomposition products comprise one or more of 5-hydroxymethylfurfural, levoglucosan and levoglucosenone; and mixtures thereof.

According to a yet further aspect of the invention there is provided a directly fermentable saccharide containing composition prepared by the microwave assisted hydrothermal treatment of the cellulosic material as herein described.

Thus, the invention further provides the use of a cellulosic material in the manufacture of a directly fermentable saccharide containing composition by the microwave assisted hydrothermal treatment of the cellulosic material.

The invention further provides a method of direct fermentation of a saccharide containing composition wherein said saccharide containing composition prepared by the microwave assisted hydrothermal treatment of the cellulosic material which comprises subjecting the saccharide containing composition to a suitable microorganism.

The method according to this aspect of the invention does not require the removal of any toxins and/or inhibitors. Furthermore, the method does not require the addition of any nutrients.

The method of direct fermentation according to this aspect of the invention is advantageous in that, inter alia, the saccharide containing mixture can be subjected to direct fermentation, without the need to remove toxins and/or inhibitors; and/or without the need to provide further nutrients to the fermentation medium.

The invention further provides the use of a saccharide containing composition in a method of direct fermentation as herein described.

In addition, the invention provides a method of producing one or more decomposition products of a monosaccharide or oligosaccharide from a cellulosic material comprising steps of:
transformation of the cellulosic material into a fermentable saccharide containing mixture by the microwave assisted hydrothermal treatment of the cellulosic material;
(ii) fermenting the fermentable saccharide containing mixture with a suitable microorganism; and
(iii) isolating the producing one or more decomposition products.

Thus, according to a yet further aspect of the invention there is provided a biorefinery for isolating components or bio-processable mixtures from cellulosic materials including the microwave assisted hydrothermal treatment of cellulosic materials as herein described.

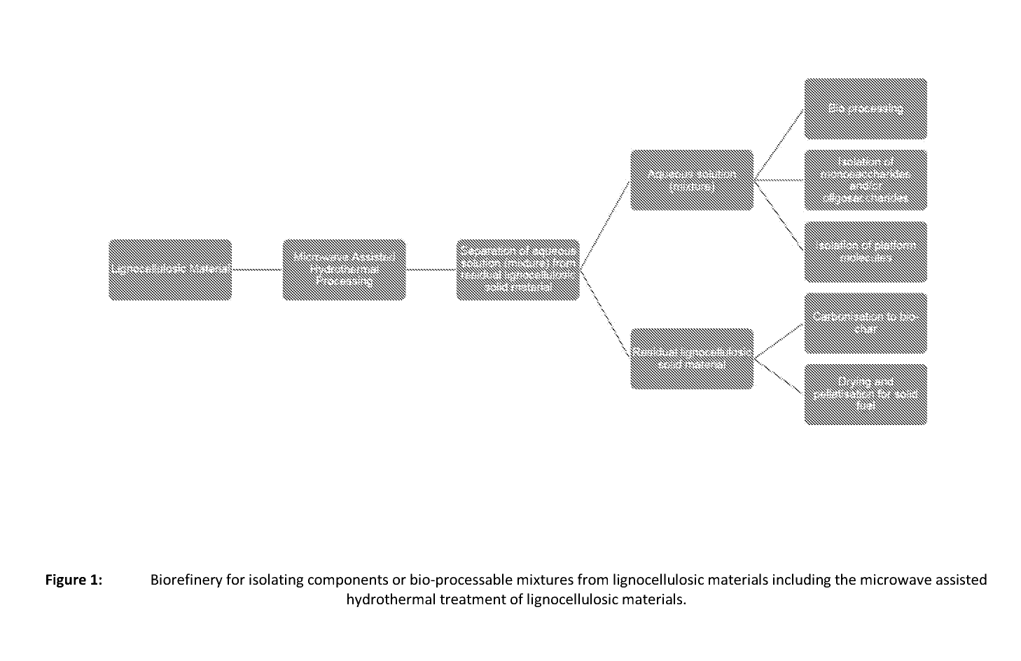

Such a biorefinery is illustrated, by way of example only, in FIG. 1 herein. Thus, according to this aspect of the invention a biorefinery process may comprise the steps of:
(i) introduction of wet cellulosic material;
(ii) subjecting the cellulosic material to microwave assisted hydrothermal treatment to produce an aqueous solution of saccharides and a cellulosic material residue;
(iii) separating the aqueous solution of saccharides and the cellulosic material residue;
(iv) subjecting the aqueous solution saccharides to bio-processing, e.g. fermentation, or isolating the saccharides; and
(v) optionally subjecting the cellulosic material residue to carbonisation to produce bio-char or drying the cellulosic material residue to produce a solid fuel.

Step (v) may include a step of pelletising the dried cellulosic material residue to produce a solid fuel.

The method of the invention is advantageous in that, inter alia, it provides a saccharide in a form suitable for fermentation with yeasts for biofuel production, for example, Metschnikowia pulcherimma yeast described in International Patent Application No. WO 2014/122439.

The present invention will now be described by way of example only with reference to the accompanying FIGURES in which:

FIG. 1 is a schematic representation of a biorefinery for isolating components or bio-processable mixtures from lignocellulosic materials including the microwave assisted hydrothermal treatment of lignocellulosic materials.

### EXAMPLE 1

### Influence of Temperature of Microwave Experiment on the Hydrolysate Composition

### Microwave Hydrolysis of Wheat Straw

The process below describes the microwave hydrolysis of wheat straw and resulting fermentable broth.

2 g of wheat straw and 40 g of deionised water were added to a microwave reactor. This was then microwaved for a period of 15 minutes, ramping to a final temperature of 190° C. with no holding time applied. The sample was allowed to cool to room temperature, at which point the solid and liquid were separated.

All the saccharides and inhibitors are identified and quantified by High Performance Liquid Chromatography (HPLC). HPLC was carried out using a Hewlett Packard Series 1100 with an Alltech 3300 Evaporative Light-Scattering Detector (ELSD). Various different columns have been used for different compound analysis.

Analysis of the hydrolysate showed that it contains very low/virtually no inhibitors, with the absence of furfural and/or HMF. The Analytical results are provided in Table 1.

TABLE 1
Chemical compounds within the wheat straw hydrolysate produced as described above (1:20 ratio, 15 mins ramping, no holding)

| | Compound Concentration g/L | | | | | | | | | | | |
|---|---|---|---|---|---|---|---|---|---|---|---|---|
| | | | | | | | | | | | Formic | Acetic |
| Temperature ° C. | Glucose | Xylose | Cellobiose | Arabinose | Arabitol | Rhamnose | Fructose | Levoglucosenon | Furfural | HMF | acid | Acid |
| 180 | 0 | 0 | 0 | 0 | 0 | 0 | | 0 | 0 | 0 | 0 | 0.01 |
| 190 | 0.64 | 0 | 0.12 | 0 | 0.04 | 0 | | 0 | 0 | 0 | 0 | 0 |
| 200 | 0.64 | 0.28 | 0.11 | 0 | 0.27 | 0 | | 0 | 0 | 0 | 0.08 | 0 |
| 210 | 0.81 | 0.3 | 0.11 | 0 | 0.5 | 0.26 | 0.01 | 0.05 | 0.007 | 0.036 | 0.09 | 0 |
| 220 | 0.76 | 0.3 | 0 | 0 | 0.01 | 0.14 | 0.01 | 0.06 | 0.033 | 0 | 0 | 0 |
| 230 | 0.63 | 0.6 | 0.14 | 0.16 | 0.2 | 0.16 | 0 | 0.07 | 0.083 | 0 | 0.11 | 0 |
| 240 | 0.81 | 0.66 | 0 | — | 0.01 | 0.23 | 0 | 0 | 0.154 | 0.086 | — | 0 |
| 250 | 0.06 | 0.23 | 0 | 0 | 0.01 | 0.07 | 0.01 | 0.04 | 0.432 | 0.104 | 0 | 0 |

### EXAMPLE 2

### Influence of Biomass Types on the Hydrolysate Composition

Further examples of different types of biomass exposed to the same microwave conditions (1:20 ratio, 15 mins ramping, no holding) are shown in Table 2. The total sugars concentration was calculated as the sum of glucose, xylose and cellobiose concentrations. The total impurity concentration has been found as a sum of furfural and HMF.

TABLE 2
Total Sugars

| | | |
|---|---|---|
| | Total amount of | Total amount of |
| Biomass | sugars (g/L) | impurities (g/L) |

| type | 190° C. | 220° C. | 190° C. | 220° C. |
|---|---|---|---|---|
| DDGS | 0.439 | 1.236 | 0.074 | 0.436 |
| DRAFF | 0.755 | 3.678 | 0.048 | 1.179 |
| RSM | 0.436 | 1.128 | 0.015 | 0.068 |
| Egg seaweed | 0.606 | 0.554 | 0.008 | 0.034 |

Where: DDGS is the Distiller's Dried Grains with Solubles hydrolysate; DRAFF is the residue from the mashing process. It consists of barley husks and other grain materials that are collected, dried and compressed in to pellets and sold as animal feed; RSM is Rape Seed Meal.

### EXAMPLE 3

### Influence of Temperature, Water to Sample Ratio and the Holding Time of Microwave Experiment on the Hydrolysate Composition

The microwave hydrolysis of pure cellulose was carried using similar conditions as Example 1. Analysis of the hydrolysate showed that it contains very low/virtually no inhibitors, with the absence of furfural and/or HMF (at a temperature lower than 220° C.). The Analytical results are provided in Tables 3 and 4.

TABLE 3
Chemical compounds within the pure cellulose hydrolysate (1:15 ratio, 15
mins ramping, with different holding time (0 min, 5 mins or 10 mins))

| | Compound Concentration g/L | | | | | | | | | | | |
|---|---|---|---|---|---|---|---|---|---|---|---|---|
| | Glucose | | | Xylose | | | Cellobiose | | | Fructose | | |
| | Holding Time (mins) | | | | | | | | | | | |
| Temperature (° C.) | 0 | 5 | 10 | 0 | 5 | 10 | 0 | 5 | 10 | 0 | 5 | 10 |
| 180 | 0 | 0 | 0 | 0 | 0 | 0 | 0 | 0 | 0 | 0 | 0 | 0 |
| 190 | 0 | 0 | 0 | 0 | 0 | 0 | 0 | 0 | 0 | 0 | 0 | 0 |
| 200 | 0 | 0 | 0.029 | 0 | 0 | 0 | 0 | 0 | 0 | 0 | 0 | 0 |
| 210 | 0.02 | 0.059 | 0.142 | 0.021 | 0 | 0.153 | 0.015 | 0 | 0.055 | 0 | 0 | 0 |
| 220 | 0.057 | 0.238 | 0.866 | 0.08 | 0.237 | 0.388 | 0.043 | 0.124 | 0.206 | 0 | 0.02 | 0.033 |
| 230 | 0.225 | 1.708 | 4.031 | 0.285 | 0.35 | 0.377 | 0.138 | 0.349 | 0.197 | 0.017 | 0.077 | 0.105 |
| 240 | 0.924 | 4.42 | 8.586 | 0.412 | 0.265 | 0.182 | 0.428 | 0.186 | 0.118 | 0.068 | 0.143 | 0.131 |
| 250 | 6.697 | 11.8 | 14.165 | 0.209 | 0.174 | 0.112 | 0.135 | 0 | 0 | 0.171 | 0.164 | 0.098 |

| | Compound Concentration g/L | | | | | | | | |
|---|---|---|---|---|---|---|---|---|---|
| | Levoglucosan | | | Furfural | | | HMF | | |
| | Holding Time (mins) | | | | | | | | |
| Temperature (° C.) | 0 | 5 | 10 | 0 | 5 | 10 | 0 | 5 | 10 |
| 180 | 0 | 0 | 0 | 0 | 0 | 0.002 | 0 | 0.003 | 0.004 |
| 190 | 0 | 0 | 0 | 0.001 | 0.003 | 0.002 | 0.002 | 0.003 | 0.008 |
| 200 | 0 | 0 | 0 | 0.003 | 0.004 | 0.004 | 0.005 | 0.012 | 0.027 |
| 210 | 0 | 0 | 0.088 | 0.008 | 0.007 | 0.004 | 0.018 | 0.043 | 0.086 |
| 220 | 0 | 0 | 0.036 | 0.022 | 0.008 | 0.009 | — | 0.126 | 0.298 |
| 230 | 0 | 0.036 | 0.105 | 0.06 | 0.028 | 0.03 | 0.119 | 0.437 | 0.941 |
| 240 | 0.012 | 0.504 | 0.356 | 0.162 | 0.035 | 0.164 | 0.414 | 1.215 | 3.08 |
| 250 | 0.265 | 1.234 | 0.524 | 0.502 | 0.086 | 0.209 | 2.142 | 4.02 | 6.6 |

TABLE 4
Chemical compounds within the pure cellulose hydrolysate (1:7 ratio, 15
mins ramping, with different holding time (0 min, 5 mins or 10 mins))

| | Compound Concentration g/L | | | | | | | | | | | |
|---|---|---|---|---|---|---|---|---|---|---|---|---|
| | Glucose | | | Xylose | | | Cellobiose | | | Fructose | | |
| | Holding Time (mins) | | | | | | | | | | | |
| Temperature (° C.) | 0 | 5 | 10 | 0 | 5 | 10 | 0 | 5 | 10 | 0 | 5 | 10 |
| 180 | 0 | 0 | 0.05 | 0 | 0 | 0 | 0 | 0 | 0 | 0 | 0 | 0 |
| 190 | 0.024 | 0.054 | 0.1 | 0.026 | 0 | 0.1 | 0 | 0 | 0.069 | 0 | 0 | 0 |
| 200 | 0.039 | 0.081 | 0.265 | 0.034 | 0.077 | 0.373 | 0.035 | 0 | 0.129 | 0 | 0 | 0 |
| 210 | 0.09 | 0.4 | 1.467 | 0.127 | 0.628 | 0.929 | 0.068 | 0.225 | 0.462 | 0.009 | 0 | 0.071 |
| 220 | 0.222 | 2.768 | 4.19 | 0.385 | 1.258 | 1.011 | 0.136 | 0.753 | 0.697 | 0.007 | 0.111 | 0.167 |
| 230 | 0.813 | 6.284 | 16.502 | 1.115 | 1.037 | 0.84 | 0.611 | 0.701 | 0.441 | 0.055 | 0.208 | 0.232 |
| 240 | 3.796 | 21.902 | 41.711 | 1.264 | 0.765 | 0.512 | 1.354 | 0.325 | 0.095 | 0.187 | 0.279 | 0.292 |
| 250 | 4.393 | 35.926 | 9.895 | 1.2 | 0 | 0 | 1.523 | 0 | 0 | 0.226 | 0.248 | 0.13 |

| | Compound Concentration g/L | | | | | | | | |
|---|---|---|---|---|---|---|---|---|---|
| | Levoglucosan | | | Furfural | | | HMF | | |
| | Holding Time (mins) | | | | | | | | |
| Temperature (° C.) | 0 | 5 | 10 | 0 | 5 | 10 | 0 | 5 | 10 |
| 180 | 0 | 0 | 0 | 0.004 | 0.003 | 0.003 | 0.003 | 0.012 | 0.027 |
| 190 | 0 | 0 | 0 | 0.005 | 0.003 | 0.005 | 0.011 | 0.035 | 0.063 |
| 200 | 0 | 0 | 0 | 0.012 | 0.005 | 0.021 | 0.034 | 0.055 | 0.173 |
| 210 | 0 | 0 | 0.019 | 0.028 | 0.006 | 0.085 | 0.075 | 0.199 | 0.425 |
| 220 | 0 | 0.052 | 0.194 | 0.075 | 0.025 | 0.035 | 0.146 | 0.611 | 1.177 |
| 230 | 0.011 | 0.313 | 0.718 | 0.195 | 0.032 | 0.05 | 0.37 | 1.372 | 3.82 |
| 240 | 0.132 | 0.953 | 1.115 | 0.374 | 0.142 | 0.482 | 1.1 | 4.62 | 11.15 |
| 250 | 0.167 | 1.045 | 0.389 | 0.433 | 0.549 | 0.481 | 1.137 | 14 | 8.8 |

### REFERENCES

(1) Alonso, D. M.; Wettstein, S. G.; Dumesic, J. A. Chemical Society Reviews 2012, 41, 8075.
(2) Hallock, J. L.; Tharakan, P. J.; Hall, C. A. S.; Jefferson, M.; Wu, W. Energy 2004, 29, 1673.
(3) Lin, Y.; Tanaka, S. Applied Microbiology and Biotechnology 2006, 69, 627.
(4) Carroll, A.; Somerville, C. Annual Review of Plant Biology 2009, 60, 165.
(5) Naik, S. N.; Goud, V. V.; Rout, P. K.; Dalai, A. K. Renewable & Sustainable Energy Reviews 2010, 14, 578.
(6) Ladisch, M. R.; Ladisch, C. M.; Tsao, G. T. Science 1978, 201, 743.
(7) Geboers, J. A.; Van de Vyver, S.; Ooms, R.; de Beeck, B. O.; Jacobs, P. A.; Sels, B. F. Catalysis Science & Technology 2011, 1, 714.
(8) Sasaki, M.; Kabyemela, B.; Malaluan, R.; Hirose, S.; Takeda, N.; Adschiri, T.; Arai, K. Journal of Supercritical Fluids 1998, 13, 261.
(9) Kobayashi, H.; Komanoya, T.; Hara, K.; Fukuoka, A. Chemsuschem 2010, 3.
(10) Mascal, M.; Nikitin, E. B. Angewandte Chemie—International Edition 2008, 47, 7924.
(11) Eyal, A.; Jansen, R.; Ltd, H. C., Ed. US, 2012.
(12) Fan, L. T.; Gharpuray, M. M.; Lee, Y. H. Cellulose Hydrolysis; Springer-Verlag: Berlin, 1987
(13) Saeman, J. F. Industrial and Engineering Chemistry 1945, 37, 43.
(14) Klyosov, A. A. Biochemistry 1990, 29, 10577.
(15) Krieger-Brockett, B. Research on Chemical Intermediates 1994, 20, 39.
(16) Lin, Y. C.; Cho, J.; Tompsett, G. A.; Westmoreland, P. R.; Huber, G. W. Journal of Physical Chemistry C 2009, 113, 20097
(17) Zhang, L., Xu, C. & Champagne, P. Overview of recent advances in thermochemical conversion of biomass. Energy Conversion and Management, 2010, 51, 969-982.
(18) Toor, S. S., Rosendahl, L. & Rudolf, A. Hydrothermal liquefaction of biomass: A review of subcritical water technologies. Energy, 36, 2328-2342.
(19) Krammer, P. & Vogel, H. Hydrolysis of esters in subcritical and supercritical water. The Journal of Supercritical Fluids, 2000, 16, 189-206.
(20) Uematsu, M. & Frank, E. U. Static Dielectric Constant of Water and Steam. Journal of Physical and Chemical Reference Data, 1980, 9, 1291-1306.
(21) A. A. Peterson, F. Vogel, R. P. Lachance, M. Froeling, M. J. Antal, Jr. and J. W. Tester, Thermochemical biofuel production in hydrothermal media: a review of sub- and supercritical water technologies, Energy & Environmental Science, 2011, 1(1) 32-65 (2008).
(22) P. Kumar, D. M. Barrett, M. J. Delwiche and P. Stroeve, Methods for pretreatment of lignocellulosic biomass for efficient hydrolysis and biofuel production, Industrial & Engineering Chemistry Research, 48(8) 3713-3729 (2009).
(23) M. von Sivers and G. Zacchi, A techno-economical comparison of three processes for the production of ethanol from pine, Bioresource Technology, 51(1) 43-52 (1995).
(24) R. Torget, M. Himmel and K. Grohmann, Dilute-acid pretreatment of two short-rotation herbaceous crops, Applied Biochemistry and Biotechnology, 34-35(1) 115-123 (1992).
(25) A. Esteghlalian, A. G. Hashimoto, J. J. Fenske and M. H. Penner, Modeling and optimization of the dilute-sulfuric-acid pretreatment of corn stover, poplar and switchgrass, Bioresource Technology, 59(2-3) 129-136 (1997).
(26) M. Galbe and G. Zacchi, Pretreatment: the key to efficient utilization of lignocellulosic materials, Biomass and Bioenergy, 46(0) 70-78 (2012).
(27) A. K. Chandel., S. S. da Silva. and O. V. Singh., Detoxification of Lignocellulosic Hydrolysates for Improved Bioethanol Production, in M. A. d. S. Bernardes (Ed.) Biofuel Production—Recent Developments and Prospects, pp. 225-246, InTech, 2011.
(28) G. Brodeur, E. Yau, K. Badal, J. Collier, K. B. Ramachandran and S. Ramakrishnan, Chemical and Physicochemical Pretreatment of Lignocellulosic Biomass: a Review, Enzyme Research, 2011 1-17 (2011).
(29) S. Haghighi Mood, A. Hossein Golfeshan, M. Tabatabaei, G. Salehi Jouzani, G. H. Najafi, M. Gholami and M. Ardjmand, Lignocellulosic biomass to bioethanol, a comprehensive review with a focus on pretreatment, Renewable and Sustainable Energy Reviews, 27(0) 77-93 (2013).
(30) Y. Chen, M. A. Stevens, Y. Zhu, J. Holmes and H. Xu, Understanding of alkaline pretreatment parameters for corn stover enzymatic saccharification, Biotechnology for Biofuels, 6(8) 1-10 (2013).
(31) J. Xu, J. J. Cheng, R. R. Sharma-Shivappa and J. C. Burns, Sodium hydroxide pretreatment of switchgrass for ethanol production, Energy & Fuels, 24(3) 2113-2119 (2010).

1. A method of transformation of a cellulosic material into a directly fermentable saccharide containing mixture wherein said method of transformation comprises the microwave assisted hydrothermal treatment of the cellulosic material.

2. A method according to claim 1 wherein the directly fermentable saccharide containing mixture, prepared by the transformation of a cellulosic material, comprises no or very low levels of toxins and/or inhibitors.

3. A method according to claim 1 or 2 wherein the cellulosic material comprises lignocellulosic biomass material.

4. A method according to claim 1 or 2 wherein the cellulosic material comprises cellulose.

5. A method according to any one of the preceding claims wherein the saccharide comprises one or more of monosaccharides, disaccharides and oligosaccharides, lignin, cellulose and/or hemi-cellulose; and mixtures thereof.

6. A method according to any one of the preceding claims wherein the saccharide comprises monosaccharides and/or oligosaccharides.

7. A method according to any one of the preceding claims wherein the fermentable saccharide containing mixture, prepared by the transformation of a cellulosic material, comprises sufficient nutrients to permit direct fermentation, or other biological treatment, to take place.

8. A method according to claim 7 wherein the cellulosic material comprises lignocellulosic biomass material.

9. A method according to claim 7 wherein the cellulosic material comprises cellulose.

10. A method according to any one of the preceding claims wherein the lignocellulosic biomass material comprises one or more of forest and/or mill residues, agricultural crops and wastes, food wastes, wood and wood wastes, agricultural waste, animal wastes, livestock operation residues, aquatic plants, fast-growing trees and plants, and municipal and industrial wastes.

11. A method according to claim 10 wherein the lignocellulosic material is waste in its origin, such as agricultural waste or forestry residue.

12. A method according to any one of the preceding claims wherein pre-existing water present in the cellulosic material is utilised.

13. A method according to claim 12 wherein the cellulosic material comprises lignocellulosic biomass material.

14. A method according to claim 12 wherein the cellulosic material comprises cellulose.

15. A method according to claim 12 which comprises the microwave steam distillation of cellulosic material in the absence of additional water.

16. A method according to claim 15 wherein the cellulosic material comprises lignocellulosic biomass material.

17. A method according to claim 15 wherein the cellulosic material comprises cellulose.

18. A method according to claim 12 which comprises the microwave assisted hydrothermal treatment of cellulosic material in the absence of additional water.

19. A method according to claim 18 wherein the cellulosic material comprises lignocellulosic biomass material.

20. A method according to claim 18 wherein the cellulosic material comprises cellulose.

21. A method according to any one of claims 1 to 14 wherein the cellulosic material is combined with water prior to being subjected to microwave energy.

22. A method according to claim 21 wherein the cellulosic material comprises lignocellulosic biomass material.

23. A method according to claim 21 wherein the cellulosic material comprises cellulose.

24. A method according to any one of the preceding claim wherein the solids-to-water ratio is from about no water to 1:50 w/w.

25. A method according to any one of claim 21 or 24 which comprises a single step.

26. A method according to any one of the preceding claims wherein the microwave irradiation power is from about 100 W to about 10 MW.

27. A method according to any one of the preceding claims whereby the cellulosic material is subjected to repeat processing by microwave assisted hydrothermal treatment.

28. A method according to claim 27 wherein the cellulosic material comprises lignocellulosic biomass material.

29. A method according to claim 27 wherein the cellulosic material comprises cellulose.

30. A method according to any one of the preceding claims which is carried out at elevated temperature and/or pressure.

31. A method according to claim 30 which is carried out at a temperature of from about 20° C. to about 300° C.

32. A method according to any one of the preceding claims which comprises the transformation of a cellulosic material to produce one or more decomposition products of a monosaccharide or oligosaccharide.

33. A method according to claim 32 wherein the cellulosic material comprises lignocellulosic biomass material.

34. A method according to claim 32 wherein the cellulosic material comprises cellulose.

35. A method according to claim 32 wherein the one or more decomposition products of a monosaccharide or oligosaccharide is selected from the group consisting of one or more of rhamnose, galactose, mannose, xylose, fructose, glucose, sucrose, cellobiose, hydroxymethylfurfural, levoglucosenone and furfural; and mixtures thereof.

36. A method according to claim 32 or 35 wherein the decomposition product comprises one or more of 5-hydroxymethylfurfural, levoglucosan and levoglucosenone; and mixtures thereof.

37. A decomposition product of a monosaccharide or oligosaccharide prepared by the method according to any one of the claims 1 to 36.

38. A decomposition product of a monosaccharide prepared by the method according to claim 37.

39. A decomposition product of a monosaccharide according to claim 38 wherein the product is 5-hydroxymethylfurfural, levoglucosan or levoglucosenone.

40. A directly fermentable saccharide containing composition prepared by the microwave assisted hydrothermal treatment of a cellulosic material.

41. A directly fermentable saccharide containing composition according to claim 40 prepared from a lignocellulosic biomass material.

42. A directly fermentable saccharide containing composition according to claim 40 prepared from cellulose.

43. The use of a cellulosic material in the manufacture of a directly fermentable saccharide containing composition by the microwave assisted hydrothermal treatment of the cellulosic material.

44. A method according to claim 1 or 2 wherein the cellulosic material comprises lignocellulosic biomass material.

45. A method according to claim 1 or 2 wherein the cellulosic material comprises cellulose.

46. A method of direct fermentation of a saccharide containing composition wherein said saccharide containing composition prepared by the microwave assisted hydrothermal treatment of the cellulosic material which comprises subjecting the saccharide containing composition to a suitable microorganism.

47. A method according to claim 46 wherein the cellulosic material comprises lignocellulosic biomass material.

48. A method according to claim 46 wherein the cellulosic material comprises cellulose.

49. A method according to claim 46 which does not require the removal of any toxins and/or inhibitors.

50. A method according to claim 46 which does not require the addition of any nutrients.

51. The use of a saccharide containing composition saccharide mixture in a method of direct fermentation according to claim 46.

52. A method of producing one or more decomposition products of a monosaccharide or oligosaccharide from a cellulosic material comprising steps of:
(i) transformation of the cellulosic material into a fermentable saccharide containing mixture by the microwave assisted hydrothermal treatment of the cellulosic material;
(ii) fermenting the fermentable saccharide containing mixture with a suitable microorganism; and
(iii) isolating the producing one or more decomposition products.

53. A method according to claim 52 wherein the cellulosic material comprises lignocellulosic biomass material.

54. A method according to claim 52 wherein the cellulosic material comprises cellulose.

55. A biorefinery process comprising the steps of:
(i) introduction of wet cellulosic material;
(ii) subjecting the cellulosic material to microwave assisted hydrothermal treatment to produce an aqueous solution of saccharides and a cellulosic material residue;
(iii) separating the aqueous solution of saccharides and the cellulosic material residue;
(iv) subjecting the aqueous solution saccharides to bio-processing, e.g. fermentation, or isolating the saccharides; and
(v) optionally subjecting the cellulosic material residue to carbonisation to produce bio-char or drying the cellulosic material residue to produce a solid fuel.

56. A method according to claim 55 wherein the cellulosic material comprises lignocellulosic biomass material.

57. A method according to claim 55 wherein the cellulosic material comprises cellulose.

58. A biorefinery process according to claim 57 wherein step (v) includes a step of pelletising the dried cellulosic material residue to produce a solid fuel.

59. A biorefinery process according to claim 57 as illustrated in FIG. 1.

60. A method, decomposition product of a monosaccharide, directly fermentable saccharide, use and biorefinery process as herein described with reference to the accompanying examples and FIGURES.
